(19) United States
(12) Patent Application Publication
(10) Pub. No.: US 2021/0346534 A1
(43) Pub. Date: Nov. 11, 2021

(54) SYSTEM AND METHOD FOR SANITIZING CURRENCY

(71) Applicant: Santrail, LLC, Mobile, AL (US)

(72) Inventors: Sheffield Clark, Southlake, TX (US); Craig Zieres, Fort Worth, TX (US)

(73) Assignee: Santrail, LLC, Mobile, AL (US)

(21) Appl. No.: 17/313,287

(22) Filed: May 6, 2021

Related U.S. Application Data
(60) Provisional application No. 63/023,053, filed on May 11, 2020.

(51) Int. Cl. A61L 2/04; A61L 2/24; G07D 11/12; G07D 11/30

(52) U.S. Cl. CPC A61L 2/04; A61L 2/24; G07D 11/12; G07D 11/30; G07D 2211/00; A61L 2202/16; A61L 2202/14; A61L 2202/181; A61L 2202/26; A61L 2202/122

(57) ABSTRACT

A system and method for sanitizing currency is provided. The system generally comprises a container, control board, switch, power supply, heating elements, temperature probe, and charging ports. Currency is inserted into the system via an entrance, and the control board provides power to the heating elements from the power supply to increase the temperature within the internal cavity of the container. The temperature probe assists the control board and heating element to maintain a desired temperature for a desired length of time in order to kill pathogens that may be living on the currency. A switch on the container may be used to complete a circuit of control board and initiate a cleaning cycle when the entrance is closed by a user.

### CROSS REFERENCES

This application claims the benefit of U.S. Provisional Application No. 63/023,053, filed on May 11, 2020, which application is incorporated herein by reference.

### BACKGROUND

Currency is a fomite, meaning it is an object or material which is likely to carry an organism that causes disease and by which said organism can be spread to others. Currency can carry viruses, protozoa, and bacteria, and has been found to carry pathogens ranging from lethal viruses, such as COVID-19, to skin irritating bacteria responsible for acne, such as propionibacterium acnes. A person can easily spread these pathogens to others by touching infected currency prior to physically contacting another person. In some instances, a person may unintentionally spread a pathogen to others by touching currency prior to touching some other fomite, such as a door handle, meaning pathogens on currency can be spread to others without even requiring person to person contact. This is particularly a problem for those who work at banks or manage ATMs since these people are at a higher risk at being exposed due to the large amount of currency handled on any given day.

Currently, there is no easy way to sanitize currency as it is deposited at a bank or ATM. Further, the World Health Organization (WHO) has advised that the best solution to deal with potentially contaminated currency is to simply wash your hands thoroughly. Though washing your hands is a good method of removing pathogens from one's own person, it does not remove the pathogen from the source, which means it can easily be transferred to others in the future. Additionally, currency often sits in buildings and transport vehicles without undergoing any type of sanitation treatment. It is inefficient to require someone to collect the currency and transfer the currency to a sanitation device prior to distributing the currency to its finally destination. This would also expose those who collect the currency prior to sanitation to pathogens.

Therefore, there is a need in the art for a system and method that sanitizes currency as it is collected without exposing those who collect it.

### SUMMARY

A system and method for sanitizing fomites is provided. In one aspect, the system allows users to sanitize currency by placing said currency in a container where heating elements heat the currency to a specified temperature. In another aspect, the system sanitizes currency using UV emitters configured to emit at wavelengths sufficient to kill organisms that cause disease. Generally, the system allows users to sanitize currency and alerts said users when a sanitation cycle has finished. The system comprises a sanitizing currency container having an internal cavity, processor operably connected to said a sanitizing currency container, power supply, and non-transitory computer-readable medium coupled to the processor and having instructions stored thereon. The processor is configured to receive temperature data and then use this information to determine when currency has been sanitized. A computing entity operably connected to the processor may comprise a user interface that may allow a user to view data of the system and/or cause the system to perform an action via commands input by said user. A database may be used to store currency data, sanitation data, and position data gathered by the system. A wireless communication interface may allow the processor to receive and transmit data of the system.

The system is designed to collect various data related to currency and sanitation cycles and save said data within user profiles so that a user may monitor the status of fomites within the sanitizing currency container. In particular, the system is designed to monitor sanitation cycles of a sanitizing currency container that may prevent the spread of unwanted disease. An at least one sensor of the sanitizing currency container may be secured within the internal cavity in a way such that it may measure temperature within said internal cavity and transmit the temperature data to the processor. The at least one sensor may be configured to measure a variety of types of sanitation data and transmit that data to the processor other than temperature. Types of sensors that may be used as the at least one sensor of the sanitizing currency container include, but are not limited to, thermometer, hygrometer, gas detector, microphone, vibration sensor, current sensor, ultrasonic sensor, infrared sensor, microwave sensor, photoelectric sensor, time-of-flight sensor, or any combination thereof.

The various data of the system may be saved within a user profile, which may be viewed within the user interface of the system. The system may perform a sanitation cycle based on sanitation cycle instructions, which may be stored within memory of the system or be received by the system in the form of a computer readable signal. A user may start, pause, or stop a sanitation cycle using functions of the user interface of a computing operably connected to the control board of the sanitizing currency container. A computing entity may be implemented in a number of different forms, including, but not limited to, servers, multipurpose computers, mobile computers, etc. Additionally, a computing entity may be made up of a single computer or multiple computers working together over a network, which may communicate via a wired or wireless connection. The user interface of the computing entity may include, but is not limited to operating systems, command line user interfaces, conversational interfaces, web-based user interfaces, zooming user interfaces, touch screens, task-based user interfaces, touch user interfaces, text-based user interfaces, intelligent user interfaces, and graphical user interfaces, or any combination thereof. The system may present data of the user interface to the user via a display operably connected to the processor.

The foregoing summary has outlined some features of the system and method of the present disclosure so that those skilled in the pertinent art may better understand the detailed description that follows. Additional features that form the subject of the claims will be described hereinafter. Those skilled in the pertinent art should appreciate that they can readily utilize these features for designing or modifying other structures for carrying out the same purpose of the system and method disclosed herein. Those skilled in the pertinent art should also realize that such equivalent designs or modifications do not depart from the scope of the system and method of the present disclosure.

### DESCRIPTON OF THE DRAWINGS

These and other features, aspects, and advantages of the present disclosure will become better understood with regard to the following description, appended claims, and accompanying drawings where:

FIG. 1 is a diagram illustrating a system embodying features consistent with the principles of the present disclosure.

FIG. 2 is a diagram illustrating a sanitizing currency container embodying features consistent with the principles of the present disclosure.

FIG. 3 is a diagram illustrating a sanitizing currency container embodying features consistent with the principles of the present disclosure.

FIG. 4 is a diagram illustrating a sanitizing currency container embodying features consistent with the principles of the present disclosure.

FIG. 5 is a diagram illustrating a sanitizing currency container embodying features consistent with the principles of the present disclosure.

FIG. 6 is a diagram illustrating the manner in which individual access to data may be granted or limited based on permission levels.

FIG. 7 is a flow chart illustrating certain method steps of a method embodying features consistent with the principles of the present disclosure.

### DETAILED DESCRIPTION

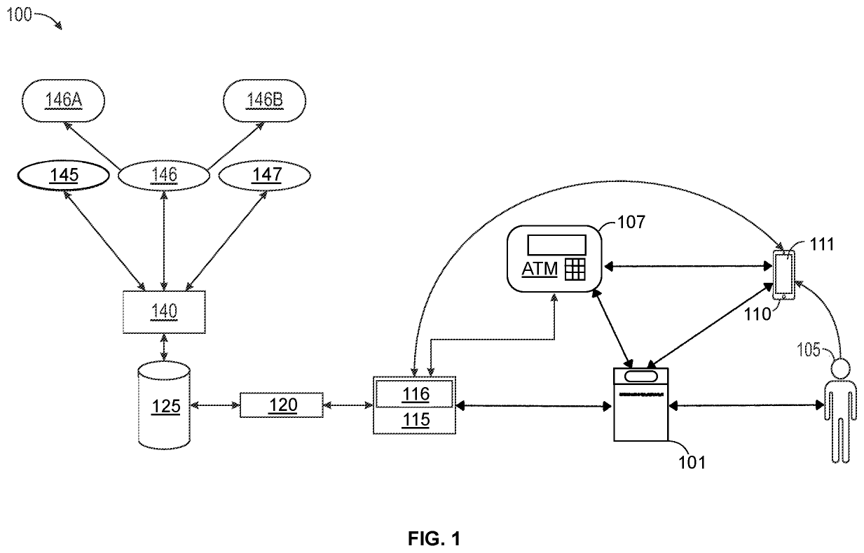

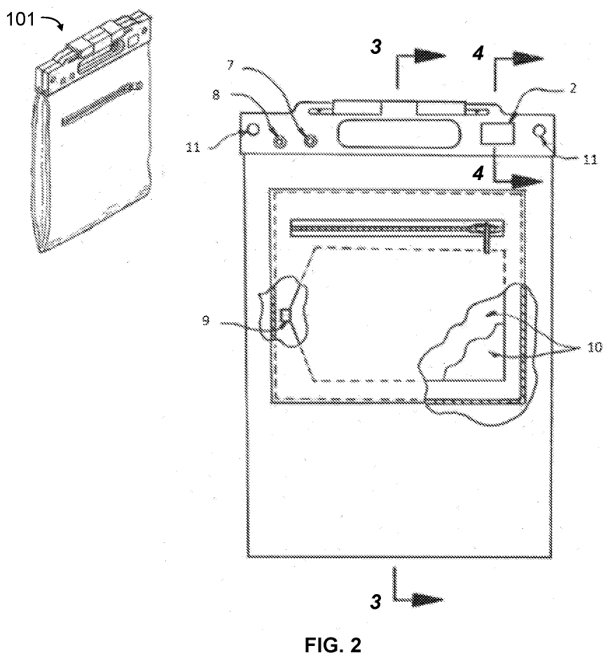

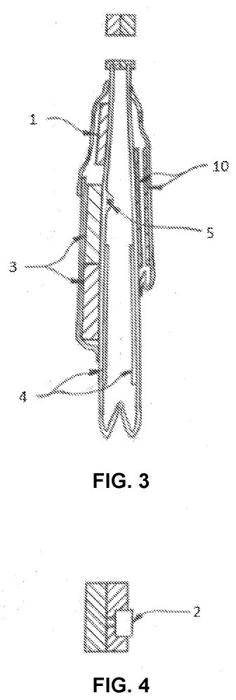

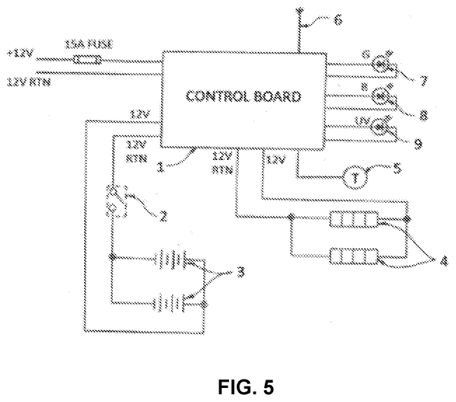

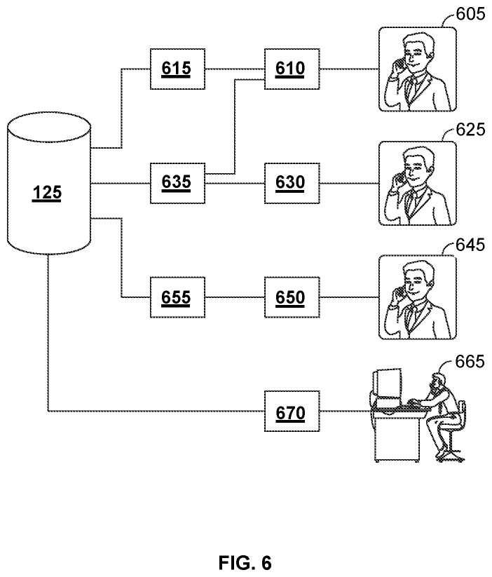

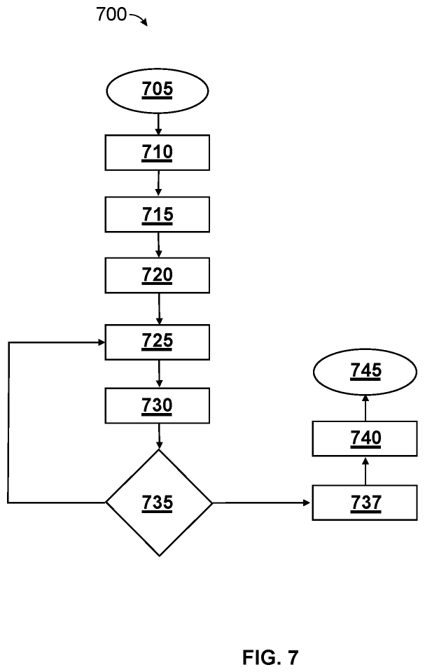

A system 100 and method for sanitizing currency is provided. Generally, the system 100 heats currency for a certain period of time at a desired temperature within a container. FIGS. 1-7 illustrate a system and methods of a system 100 used to sanitize currency. FIG. 1 depicts a preferred embodiment of a system 100 designed to sanitize currency. FIG. 2 depicts a front sectional view of a preferred embodiment of a sanitizing currency container 101, including ultraviolet (UV) emitters 9 used in a secondary cleaning process of the system 100. FIG. 3 depicts a side sectional view of a preferred embodiment of a sanitizing currency container 101, including UV emitters 9 used in a secondary cleaning process of the system 100. FIG. 4 depicts a preferred embodiment of a switch 2 that may be used to start/stop the sanitizing cycles of the sanitizing currency container 101 when it is opened and/or closed. FIG. 5 depicts the preferred embodiment of a control board 1 designed to control the various features of the sanitizing currency container 101. FIG. 6 illustrates permission levels 600 that may be utilized by the present system 100 for controlling access to content 615, 635, 655 of the system 100. FIG. 7 illustrates a method that may be carried out by a user using the system 100. It is understood that the various method steps associated with the methods of the present disclosure may be carried out using the sanitizing currency container 101 depicted in FIGS. 1-6.

As illustrated in FIG. 1, the system generally comprises a sanitizing currency container 101 having an internal cavity, processor 115 operably connected to said a sanitizing currency container 101, power supply, and non-transitory computer-readable medium 116 coupled to the processor 115 and having instructions stored thereon. In one embodiment, the system 100 may comprise a computing device 110, which may include one or more ATMs 107. The computing device 110 may comprise a user interface 111 that may allow a user 105 to view data of the system 100 and/or cause the system 100 to perform an action via commands input by said user 105. In another embodiment, the system 100 may comprise a database 125 operably connected to the processor 115, which may be used to store the various data of the system 100 therein. In a preferred embodiment, the data is stored within user profiles 140 of the system 100. In yet another preferred embodiment, a server 120 may be operably connected to the processor 115 and database 125, facilitating the transfer of information between the processor 115 and/or database 125. The system 100 preferably transmits the various data of the system 100 to a user interface 111 of a user's 105 computing device 110 to so that it may be presented to said user 105.

The sanitizing currency container 101 generally comprises a container, control board 1, switch 2, power supply 3, heating elements 4, at least one sensor 5, and charging ports 11. Other preferred embodiments may further comprise at least one LED and/or a UV emitting device affixed to light defusing waveguides 10. Generally, currency is inserted into an internal cavity of the device that may be heated by heating elements 4 at a temperature known to kill bacteria and/or viruses. Heating elements 4 within the container increase the temperature until the at least one sensor 5 determines that the desired temperature has been reached. The control board 1 then regulates the flow of power to the heating elements 4 from the power supply 3 to maintain the desired temperature until a desired period of time has elapsed. Once the sanitizing cycle is complete, a switch 2 of the control board 1 may break the circuit, thus ceasing the flow of power to the heating elements 4 and preventing them from continuing heating the internal cavity. In a preferred embodiment, the desired temperature and length of time currency is heated in the sanitizing currency container 101 depends on the pathogen desired to be destroyed. For instance, the sanitizing currency container 101 may be programmed to heat currency at a higher temperature and for a longer period of time when eradicating bacteria than it might otherwise do when programmed to eradicate viruses.

The container holds currency and houses the various components of the sanitizing currency container 101. In a preferred embodiment, the container is a pouch and comprises an insulative material that forms at least one internal cavity. Types of insulative material that may be used to make the container include, but are not limited to, polybenzimidazole fiber, aramids, fire resistant cotton, melamine, modacrylic, leather, or any combination thereof. In one preferred embodiment, the container may comprise a heat resistant material lining the inner cavity and a non-heat resistant material lining the exterior of the container. For instance, the container may comprise of polyester and leather, wherein the leather and polyester are sewn together in a way such that the interior cavity walls are made of leather and the exterior surface of the container is made of polyester. As illustrated in FIG. 2, the container may further comprise a handle that may be used to hang the device while in transit. In some preferred embodiments, the handle may also house positive and negative charging ports 11 that may be used to provide power to the sanitizing currency container 101. Multiple containers may be hung on common rods, providing a compact means to stow and retain the containers during transportation.

The control board 1, as illustrated in FIG. 5 comprises at least one circuit and microchip. In another preferred embodiment, the control board 1 may further comprise a GPS chip, which may allow the control board 1 to report position data 147 in addition to access data relating to the opening and closing of the container as well as sanitation data 146 relating to temperature data 146A the sanitation cycle instruction 146B of the system 100. The control board 1 may regulate the transfer of power to the various elements of the device and control the temperature of the internal cavity, detect the presence of currency, and control sanitizing cycles by regulating the power transferred to the heating elements 4 and UV emitters 9. The microchip of the control board 1 comprises a microprocessor, antenna 6, and memory. The microprocessor may be defined as a multipurpose, clock driven, register based, digital-integrated circuit which accepts binary data as input, processes it according to instructions stored in its memory, and provides results as output. In a preferred embodiment, the microprocessor may receive a signal to start the cleaning process from a switch 2 operably connected to the control board 1, wherein the switch 2 completes a circuit of the control board 1 when an entrance of the container is closed by a user. Alternatively, the microprocessor may receive instructions from a computing device 110 via an antenna 6 to start a sanitation cycle, wherein the instructions for said sanitation cycle is saved within the memory. For instance, a communication device may transmit instructions to the microprocessor of the microchip via the antenna 6 that cause the microprocessor to retrieve sanitation cycle instructions 146B from the memory that instruct the processor in how to carry out said sanitation cycle.

Memory may be defined as a device capable of storing information permanently or temporarily. In the preferred embodiment, memory of the microchip stores information pertaining currency and sanitation cycles within. This data may include, but is not limited to, date currency was received, currency amount, date container was retrieved, date currency was sanitized, sanitation cycle instructions 146B (including temperature and duration), etc. In some preferred embodiments, memory may include one or more volatile memory units. In another preferred embodiment, memory may include one or more non-volatile memory units. A memory device may refer to storage space within a single storage device or spread across multiple storage devices. Types of devices that may act as memory may include, but are not limited to, read only memory (ROM), random access memory (RAM), and flash memory. ROM may comprise a conventional ROM device or another type of static storage device that stores static information and instructions for execution by the microprocessor. RAM may comprise a conventional RAM device or another type of dynamic storage device that stores information and instructions for execution by the processor.

As illustrated in FIG. 4, the antenna 6 is operably connected to the processor in a way such that it may transmit information received from a computing device 110 to the processor. The antenna 6 may also be used by the processor to transmit data back to the computing device 110. In one preferred embodiment, the antenna 6 may receive electromagnetic energy from a computing device 110 and convert that energy into electricity that may be used to power the microchip. The shape of the antenna 6 may differ depending on the frequency at which the antenna 6 is to operate. Shapes in which the antenna 6 may be formed include, but are not limited to, spiral coil, single dipole, dual dipoles, and folded dipole, or any combination thereof. The antenna 6 preferably comprises copper, aluminum, or silver. However, one with skill in the art will appreciate that the antenna 6 may comprise of any material that may allow it to transmit and receive radio waves.

In a preferred embodiment, the at least one sensor 5 is a temperature probe designed to collect temperature data 146A from within the internal cavity of the container. The temperature probe 5 is preferably attached to the interior wall of the inner cavity. Alternatively, the temperature probe 5 may be at least partially imbedded inside of the material that makes up an interior wall of the interior cavity. In yet another preferred embodiment, the temperature probe 5 may be embedded within a piece of heat resistant material within the internal cavity that is separate from the material that makes up the interior walls of the interior cavity. Types of at least one sensors 5 that may act as temperature probes within the sanitizing currency container 101 include, but are not limited to, thermocouples, resistive temperature measuring devices, infrared sensors, bimetallic devices, thermometers, or any combination thereof. In one preferred embodiment, the at least one sensors 5 may detect the presence of currency within the internal cavity by measuring temperature rise over time due to the higher specific heat of the contents over air. In another preferred embodiment, a computing device 110 operably connected to the control board 1 may transmit a computer readable signal to the processor when currency is added to the internal cavity. For instance, as illustrated in FIG. 2, a sanitizing currency container 101 operably connected to an ATM 107 may receive currency data 145 from the ATM 107, wherein said currency data includes information such as the amount of currency deposited, time of deposits, etc. The ATM 107 may also transmit sanitation cycle instructions 146B to the sanitizing currency container 101, which may cause the system 100 to begin a sanitation cycle.

The internal cavity of the container is preferably heated by heating elements 4, which are operably connected to the control board 1 in a way such that they may receive power from a power supply 3. Types of heating elements 4 that may be used in the sanitizing currency container 101 include, but are not limited to, resistance wire, ceramic heaters, radiant heaters, or any combination thereof. In a preferred embodiment, the heating elements 4 increase in temperature due to resistive heating in which resistance within the heating elements 4 causes an increase in temperature of the heating elements 4 as electricity is passed through. The power supply 3 may be connected to the control board 1 in a way such that the control board 1 may regulate the amount of power the heating elements 4 receive, thus granting the control board 1 the ability to control the temperature within the cavity of the container. Types of power supplies 3 that may supply the sanitizing currency container 101 with power include, but are not limited to, batteries, wall outlets, alternators, circuit boards, or any combination thereof.

In the preferred embodiment, the power supply 3 is a battery, as illustrated in FIGS. 2 and 3. In some preferred embodiments, the batteries may be removable to ease replacement. In other preferred embodiments, the batteries may be charged via a standard bi-polar jack as an option to charging through ports 11. The batteries may be on board the sanitizing currency container 101 or part of another device operably connected to the sanitizing currency container 101. For instance, an ATM 107 that accepts deposits may provide power to the sanitizing currency container 101 via an onboard battery of an ATM 107 circuit board operably connected to the switch 2. Alternatively, the system 100 may have no battery. For instance, the sanitizing currency container's 101 charging port may be operably connected to a circuit board of a computing device 110, which may provide power thereto. For instance, the sanitizing currency container's 101 charging port may be operably connected to an alternator of an armored truck via an outlet extending from the alternator to the interior of the armored truck.

In one preferred embodiment, the system 100 may comprise multiple power supplies 3. For instance, the sanitizing currency container 101 may be optimized to receive power from a battery and a second power source, such as a wall outlet or alternator. The control board 1 may cause the second power source to charge the batteries of the system 100 while concurrently providing power to the sanitizing currency container 101. Once the batteries have been charged, the control board 1 may be configured to cause the second power source to cease charging the batteries while continuing to supply the system 100 with power. In this way, battery health of the sanitizing currency container 101 may be maintained and the sanitizing currency container 101 may continuously receive its power from the second power source while connected thereto, which may extend the life of batteries of the system 100. Once the sanitizing currency container 101 is disconnected from the second power source, the control board 1 may cause the system 100 to be powered using the the batteries, allowing the system 100 to sanitize currency in situations where mobility is desired.

In some preferred embodiments, the at least one circuit of the control board 1 may be incomplete, requiring an additional circuit piece to complete the at least one circuit and start a sanitation cycle. The switch 2, illustrated in FIG. 4, may complete a circuit of the control board 1, as illustrated in FIG. 5, thus allowing the transfer of power from the power supply 3 to the heating elements 4. In one preferred embodiment, the switch 2 may be connected to the container in a way such that closing an entrance of the container causes the switch 2 to close a circuit of the control board 1, thus allowing the heating elements 4 to heat the interior cavity. In another preferred embodiment, the control board 1 may access the power supply 3 via the charging ports 11 and may open and close the circuit as needed to regulate the flow of power to the heating elements 4 in order to maintain a desired temperature.

As illustrated in FIGS. 2 and 3, some preferred embodiments of the sanitizing currency container 101 may include an internal cavity that comprises a UV emitter affixed to light defusing waveguides 10. In a preferred embodiment, the UV radiation emitted by the UV emitter is generally between 200 and 300 nanometers; however, other wavelengths may be desirable depending on the bacteria/virus desired to be eradicated. The light defusing waveguides 10 may provide the cavity with uniform and complete radiation within the enclosure, which may be used in addition to or separate from the thermal sanitation process discussed above. In the preferred embodiment, the UV emitter and light defusing waveguides 10 are located within an internal cavity that is separate from the internal cavity comprising a temperature probe 5 and heating elements 4; however, some preferred embodiments may comprise a cavity having a temperature probe 5, heating elements 4, and UV emitters 9 in a single cavity. Types of devices that may act as waveguides include, but are not limited to, strip waveguides, rib waveguides, segmented waveguides, laser-inscribed waveguides, and optical fiber, or any combination thereof

Some preferred embodiments of the system 100 may comprise at least one light emitting diode (LED) 7 and 8, which may be used to alert a user 105 of the presence of currency within the container. In another preferred embodiment, a plurality of LEDs 7 and 8 may be used to indicate the beginning and completion of a heating cycle. LEDs 7 and 8 may also be used to alert when the contents of the internal cavity have reached a temperature that may be safe for a user 105 to handle. For instance, the sanitizing currency container 101 may comprise an LED 7 and 8 that alerts a user 105 when the device is undergoing the heating sanitation process, an LED 7 and 8 that alerts a user 105 when the device has finished the heating sanitation process, and an LED 7 and 8 that alerts a user 105 when the currency within the device has reached a temperature that is safe to handle.

As mentioned previously, some preferred embodiments of the system 100 may further comprise a user interface 111. A user interface 111 may be defined as a space where interactions between a user 105 and the system 100 may take place. In a preferred embodiment, the interactions may take place in a way such that a user 105 may control the operations of the system 100, and more specifically, allow a user 105 to monitor data and sanitation cycles of the system 100. A user 105 may input instructions to control operations of the system 100 manually using an input device. For instance, a user 105 may choose to begin or cancel sanitation cycles of the system 100 by using an input device of the system 100, including, but not limited to, a keyboard, mouse, or touchscreen. A user interface 111 may include, but is not limited to operating systems, command line user interfaces, conversational interfaces, web-based user interfaces, zooming user interfaces, touch screens, task-based user interfaces, touch user interfaces, text-based user interfaces, intelligent user interfaces, and graphical user interfaces, or any combination thereof. The system 100 may present data within the user interface 111 to the user via a display operably connected to the processor 115.

In another preferred embodiment, the user interface 111 may comprise an emergency button that may allow the user 105 to alert third parties of an emergency situation. Other preferred embodiments of the system 100 may comprise a physical switch located on the sanitizing currency container 101 that may be activated to cause the system to alert third-parties of an emergency situation. For instance, a police department may be alerted of an attempted theft via the emergency button or the physical switch and then use position data 147 of the system 100 to dispatch the emergency personnel to the user's 105 geolocation. In another preferred embodiment, the user interface 111 may comprise a start, stop, and pause button that may cause the system to start, stop, and pause a sanitation cycle for a specified period of time. For instance, a user 105 needing to place additional currency within an internal cavity of a sanitizing currency container 101 that is in the middle of a sanitation cycle may want to pause the sanitation process just long enough to put the new currency therein instead of stopping the sanitation cycle and starting the process all over.

In a preferred embodiment, users 105 may access data of the system 100 via the user interface 111, which may be accomplished by causing the processor 115 to query the non-transitory computer-readable medium 116 and/or database 125. The non-transitory computer-readable medium 116 and/or database 125 may then transmit data back to the processor 115, wherein the processor 115 may present it to the user 105 via a display. In one preferred embodiment, the user interface 111 of the system 100 may allow a user 105 to direct how the system 100 performs a sanitation cycle. For instance, the system 100 may be configured to alert a user 105 five minutes prior to the end of a sanitation cycle. For instance, the system 100 may be configured to change the temperature range depending on the fomite inserted therein. Therefore, some preferred embodiments of the system 100 may comprise a user interface 111 that may present data while also allowing a user 105 to control the various features of the sanitizing currency container 101.

Some embodiments of the system 100 may comprise a server 120. A server 120 may be a search server, a document indexing server, and general web server. Servers 120 may be separate entities performing different functions or similar functions. For instance, two or more servers 120 may be implemented to work as a single server performing the same tasks. Alternatively, one server may perform the functions of multiple servers. For instance, a single server may perform the tasks of a web server and an indexing server. Although represented as a single server in FIG. 1, it is understood that multiple servers 120 may be used to operably connect the processor 115 to the database 125. The processor 115 may be operably connected to the server 120 via wired or wireless connection. Search servers may include one or more computing devices 110 designed to implement a search engine, such as a documents/records search engine, general webpage search engine, etc. Search servers, for example, may include one or more web servers to receive search queries and/or inputs from users 105, search one or more databases 125 in response to the search queries and/or inputs, and provide documents or information, relevant to the search queries and/or inputs, to users 105. In some implementations, search servers may include a web search server that may provide webpages to users 105, where a provided webpage may include a reference to a web server at which the desired information and/or links is located. The references, to the web server at which the desired information is located, may be included in a frame and/or text box, or as a link to the desired information/document.

Document indexing servers may include one or more computing devices 110 designed to index documents available through networks. Document indexing servers may access other servers, such as web servers that host the system 100, to index the data of the system. In some implementations, document indexing servers may index documents/records stored by other servers 120 connected to the network. Document indexing servers may, for example, store and index currency data 145, sanitation data 146, position data 147, and other information relating to sanitizing currency. Web servers may include servers 120 that provide webpages to clients. For instance, the webpages may be HTML-based webpages. A web server may host one or more websites. A website, as the term is used herein, may refer to a collection of related webpages. Frequently, a website may be associated with a single domain name, although some websites may potentially encompass more than one domain name. The concepts described herein may be applied on a per-website basis. Alternatively, in some implementations, the concepts described herein may be applied on a per-webpage basis.

To prevent un-authorized users 105 from accessing data within the user profiles 140 of the system 100, the system 100 may employ a security method. As illustrated in FIG. 6, the security method of the system 100 may comprise a plurality of permission levels 600 that may allow a user 105 to view content 615, 635, 655 within the database 125 while simultaneously denying users 105 without appropriate permission levels 600 the ability to view said content 615, 635, 655. To access the data stored within the database 125, users 105 may be required to make a request via a user interface 111. Access to the data within the database 125 may be granted or denied by the processor 115 based on verification of a requesting user's 605, 625, 645 permission level. If the requesting user's 605, 625, 645 permission level 600 is sufficient, the processor 115 may provide the requesting user 605, 625, 645 access to content 615, 635, 655 stored within the system 100. Conversely, if the requesting user's 605, 625, 645 permission level 600 is insufficient, the processor 115 may deny the requesting user 605, 625, 645 access to content 615, 635, 655 stored within the system 100. In an embodiment, permission levels 600 may be based on user roles 610, 630, 650 and administrator roles 670, as illustrated in FIG. 6. User roles 610, 630, 650 allow users to access content 615, 635, 655 that a user has uploaded and/or otherwise obtained through use of the system 100. Administrator roles 670 allow administrators 665 to access system 100 wide data, including managerial permissions, as well as assign new tasks to other users.

In an embodiment, user roles 610, 630, 650 may be assigned to a user in a way such that a requesting user 605, 625, 645 may access user profiles 140 via a user interface 111. To access the data within the database 125, a user may make a user request via the user interface 111 to the processor 115. In an embodiment, the processor 115 may grant or deny the request based on the permission level 600 associated with the requesting user 605, 625, 645 assigned via user roles 610, 630, 650. Only users 105 having appropriate user roles 610, 630, 650 or administrator roles 670 may access the content 615, 635, 655. For instance, as illustrated in FIG. 6, requesting user 1 605 has a permission level 600 to view user 1 content 615 whereas requesting user 2 625 has a permission level 600 to view user 1 content 615, user 2 content 635, and user 3 content 655. Alternatively, content 615, 635, 655 may be restricted in a way such that a user 105 may only view a limited amount of content 615, 635, 655. For instance, requesting user 3 645 may be granted a permission level 600 that only allows them to view user 3 content 655 related to a particular sanitizing currency container 101. Therefore, the permission levels 600 of the system 100 may be assigned to users 105 in various ways without departing from the inventive subject matter described herein.

FIG. 7 provides a flow chart 700 illustrating certain, preferred method steps that may be used to carry out the method of sanitizing currency using the sanitizing currency container 101. Step 705 indicates the beginning of the method. During step 710, a user 105 may obtain a device configured to receive currency deposits via a sanitizing currency container 101. In a preferred embodiment, the device configured to receive currency deposits is an ATM 107. The user 105 may remove the sanitizing currency container 101 from the device configured to receive currency deposits during step 715. Once removed, the user 105 may close an entrance of the sanitizing currency container 101 during step 720, which may instruct the sanitizing currency container 101 to start a sanitation cycle. In a preferred embodiment, a switch 2 may instruct the control board 1 to begin a sanitation cycle when the user 105 closes said entrance of the sanitizing currency container 101. The user 105 may then store the sanitizing currency container 101 in a secure location and subsequently perform a query to check the status of the sanitation cycle of the sanitizing currency container 101 during steps 725 and 730, respectively. In a preferred embodiment, an LED 7 and 8 of the sanitizing currency container 101 may indicate if the device is currently undergoing or finished with a sanitation cycle.

Based on the results of the query, a user 105 may take an action during step 735. If the user 105 determines that the sanitation cycle is not complete, the user 105 may return to step 725. If the user 105 determines that the sanitation cycle is complete, the user 105 may open the sanitizing currency container 101 and remove the currency during step 737. Once the currency has been removed from the sanitizing currency container 101, the user 105 may reinstall the sanitizing currency container 101 inside the device configured to receive currency deposits during step 740. The user 105 may then proceed to the terminate method step 745. In some preferred embodiments, a user 105 may install a new sanitizing currency container 101 within the device configured to receive currency deposits immediately after removal of the originally installed sanitizing currency container 101, thus allowing the user 105 to remain mobile while the sanitizing currency container 101 sanitizes the currency within.

The subject matter described herein may be embodied in systems, apparati, methods, and/or articles depending on the desired configuration. In particular, various implementations of the subject matter described herein may be realized in digital electronic circuitry, integrated circuitry, specially designed application specific integrated circuits (ASICs), computer hardware, firmware, software, and/or combinations thereof. These various implementations may include implementation in one or more computer programs that may be executable and/or interpretable on a programmable system including at least one programmable processor, which may be special or general purpose, coupled to receive data and instructions from, and to transmit data and instructions to, a storage system, and at least one peripheral device.

These computer programs, which may also be referred to as programs, software, applications, software applications, components, or code, may include machine instructions for a programmable processor, and may be implemented in a high-level procedural and/or object-oriented programming language, and/or in assembly machine language. As used herein, the term “non-transitory computer-readable medium” refers to any computer program, product, apparatus, and/or device, such as magnetic discs, optical disks, memory, and Programmable Logic Devices (PLDs), used to provide machine instructions and/or data to a programmable processor, including a non-transitory computer-readable medium that receives machine instructions as a computer-readable signal. The term “computer-readable signal” refers to any signal used to provide machine instructions and/or data to a programmable processor. To provide for interaction with a user, the subject matter described herein may be implemented on a computer having a display device, such as a cathode ray tube (CRD), liquid crystal display (LCD), light emitting display (LED) monitor for displaying information to the user and a keyboard and a pointing device, such as a mouse or a trackball, by which the user may provide input to the computer. Displays may include, but are not limited to, visual, auditory, cutaneous, kinesthetic, olfactory, and gustatory displays, or any combination thereof.

Other kinds of devices may be used to facilitate interaction with a user as well. For instance, feedback provided to the user may be any form of sensory feedback, such as visual feedback, auditory feedback, or tactile feedback; and input from the user may be received in any form including, but not limited to, acoustic, speech, or tactile input. The subject matter described herein may be implemented in a computing system that includes a back-end component, such as a data server, or that includes a middleware component, such as an application server, or that includes a front-end component, such as a client computer having a graphical user interface or a Web browser through which a user may interact with the system described herein, or any combination of such back-end, middleware, or front-end components. The components of the system may be interconnected by any form or medium of digital data communication, such as a communication network. Examples of communication networks may include, but are not limited to, a local area network (“LAN”), a wide area network (“WAN”), metropolitan area networks (“MAN”), and the internet.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For instance, the implementations described above can be directed to various combinations and subcombinations of the disclosed features and/or combinations and subcombinations of several further features disclosed above. In addition, the logic flow depicted in the accompanying figures and/or described herein do not necessarily require the particular order shown, or sequential order, to achieve desirable results. It will be readily understood to those skilled in the art that various other changes in the details, devices, and arrangements of the parts and method stages which have been described and illustrated in order to explain the nature of this inventive subject matter can be made without departing from the principles and scope of the inventive subject matter.

What is claimed is:

1) A system for sanitizing fomites comprising:
a container having an internal cavity,
wherein interior walls of said container comprises a heat resistant material,
a heating element situated within said internal cavity,
wherein said heating element increases an internal temperature within said internal cavity,
a control board operably connected to said heating element,
wherein said control board controls a sanitation cycle designed to sanitize a fomite placed within said internal cavity,
a sensor operably connected to said control board,
wherein at least one of said sensor is configured to collect temperature data related to an internal temperature of said internal cavity,
a power supply operably connected to said control board,
wherein said power supply supplies power to said heating element in a way that causes said heating element to heat said internal cavity.

2) The system of claim 1, further comprising a switch having a first circuit and a second circuit,
wherein said switch is operably connected to said control board,
wherein said switch connects said power supply to said heating element,
wherein said first circuit and second circuit are positioned opposite one another on an entrance of said container,
wherein closing said entrance causes said first circuit and said second circuit to form a complete circuit,
wherein said power supply supplies said power to said heating element via said complete circuit.

3) The system of claim 2, wherein said switch sends a computer readable signal to said control board when said complete circuit is formed.

4) The system of claim 1, further comprising a computing device operably connected to said control board,
wherein said computing device transmits a computer readable signal to said control board that controls said sanitation cycle.

5) The system of claim 4, further comprising a user interface of said computing device,
wherein said user interface allows a user to begin said sanitation cycle,
wherein said user interface allows said user to stop said sanitation cycle.

6) The system of claim 5, wherein said computing device is configured to collect currency data related to said fomite, wherein said currency data is presented to said user via said user interface.

7) The system of claim 1, further comprising a global positioning system (GPS) sensor operably connected to said control board,
wherein said GPS sensor is configured to collect position data related to a position of said container having said fomite contained therein.

8) The system of claim 7, wherein a user interface of a computing device is configured to display said position on a GIS, wherein currency data of said container is displayed with said position on said GIS.

9) The system of claim 1, further comprising a non-transitory computer-readable medium coupled to said control board and having instructions stored thereon, which, when executed by said control board, cause said control board to perform operations comprising:
receiving a computer readable signal containing sanitation cycle instructions,
beginning said sanitation cycle based on said sanitation cycle instructions,
receiving temperature data from said sensor,
controlling said power transferred from said power supply to said heating element based on said temperature data and said sanitation cycle instructions,
stopping said sanitation cycle based on said sanitation cycle instructions.

10) A system for sanitizing fomites comprising:
a container having an internal cavity,
wherein interior walls of said container comprises a heat resistant material,
a processor operably connected to a heating element,
wherein said heating element is situated within said internal cavity,
wherein said heating element increases an internal temperature within said internal cavity,
a sensor operably connected to said processor,
wherein at least one of said sensor is configured to collect temperature data related to an internal temperature of said internal cavity,
a power supply operably connected to said processor and said heating element,
a non-transitory computer-readable medium coupled to said processor and having instructions stored thereon, which, when executed by said processor, cause said processor to perform operations comprising:
receiving a computer readable signal containing sanitation cycle instructions,
beginning a sanitation cycle based on said sanitation cycle instructions,
receiving temperature data from said sensor,
controlling power transferred from said power supply to said heating element based on said temperature data and said sanitation cycle instructions,
stopping said sanitation cycle based on said sanitation cycle instructions.

11) The system of claim 10, further comprising at least one circuit operably connected to said processor,
wherein said at least one circuit connects said power supply to said heating element,
wherein said at least one circuit can be placed in an on position and an off position,
wherein said power supply supplies said power to said heating element when in said on position,
wherein said power supply cannot supply said power to said heating element when in said off position.

12) The system of claim 11, wherein said processor is operably connected to said at least one circuit in a way such that it places said at least one circuit in said on position and said off position.

13) The system of claim 12, further comprising a computing device operably connected to said processor,
wherein said processor receives a computer readable signal from said computing device that causes said processor to place said at least one circuit in one of said on position and said off position.

14) The system of claim 13, further comprising a user interface of said computing device,
wherein said user interface allows a user to start said sanitation cycle,
wherein said user interface allows said user to stop said sanitation cycle.

15) The system of claim 13, wherein said computing device is configured to collect currency data related to a fomite, wherein said currency data is presented to a user via a user interface of said computing device.

16) The system of claim 10, further comprising a global positioning system (GPS) sensor operably connected to said processor,
wherein said GPS sensor is configured to collect position data related to a position of said container having a fomite contained therein.

17) The system of claim 16, wherein a user interface is configured to display said position on a GIS, wherein currency data of said container is displayed with said position on said GIS.

18) A method for sanitizing fomites via a sanitizing container comprising the steps of:
obtaining a sanitizing container,
wherein said sanitizing container contains a fomite within an internal cavity,
closing an entrance of said sanitizing container,
wherein closing said entrance causes a switch of said sanitizing container to close a circuit,
wherein closing said circuit allows a power supply operably connected to a control board to transfer power to a heating element,
wherein closing said circuit initiates a sanitation cycle of said control board,
waiting for said sanitation cycle to finish,
opening said entrance of said sanitizing container once said sanitation cycle has finished, and
removing said fomite from said sanitizing container.

19) The method of claim 18, further comprising the steps of:
obtaining a computing device having a user interface and operably connected to said control board,
selecting a stop function of said user interface that causes said control board to stop said sanitation cycle.

20) The method of claim 19, further comprising the steps of:
monitoring said user interface to view sanitation data collected by a sensor located within said internal cavity of said container.
